(19) United States
(12) Patent Application Publication
(10) Pub. No.: US 2013/0139273 A1
(43) Pub. Date: May 30, 2013

(54) Chronic Lymphocytic Leukemia Modeled in Mouse by Targeted miR-29 Expression

(71) Applicants: Carlo M. Croce, Columbus, OH (US); Yuri Pekarsky, Columbus, OH (US)

(72) Inventors: Carlo M. Croce, Columbus, OH (US); Yuri Pekarsky, Columbus, OH (US)

(73) Assignee: THE OHIO STATE UNIVERSITY, Columbus, OH (US)

(21) Appl. No.: 13/805,860

(22) PCT Filed: Jun. 20, 2011
(86) PCT No.: PCT/US11/41046

§ 371 Date: Jan. 18, 2013

Related U.S. Application Data
(60) Provisional application No. 61/358,383, filed on Jun. 24, 2010.

(51) Int. Cl. A01K 67/027

(52) U.S. Cl. CPC A01K 67/0278
USPC 800/3; 800/13; 800/18; 800/9; 435/29

(57) ABSTRACT

A mouse model and uses there of for detecting, treating, characterizing, and diagnosing various diseases are described.

### CROSS-REFERENCE TO RELATED APPLICATIONS

This is a national stage application filed under 37 CFR 1.371 of international application PCT/US20xx/xxxxxx filed xxx, xx, xxxx which claims the priority to United States Provisional Application Ser. No. 61/358,383 filed Jun. 24, 2010, the entire disclosures of which are expressly incorporated herein by reference.

### STATEMENT REGARDING FEDERALLY SPONSORED RESEARCH

This invention was made with government support under Grant No. P01-CA81534 warded by the National Institutes of Health. The government has certain rights in the invention.

### SEQUENCE LISTING

The instant application contains a Sequence Listing which has been submitted in ASCII format via EFS-Web and is hereby incorporated by reference in its entirety. Said ASCII copy, created on Jun. 17, 2011, is named 604_{—}52020_Seq_List_OSU-10162.txt and is 1,399 bytes in size.

TECHNICAL FIELD AND INDUSTRIAL APPLICABILITY OF THE INVENTION

The present invention relates to a mouse model and uses thereof for detecting, treating, characterizing, and diagnosing various diseases.

### BACKGROUND

Chronic lymphocytic leukemia (CLL) is the most common human leukemia, accounting for −30% of all cases, with 10,000 new cases observed each year in the United States. Characteristically, CLL is a disease of elderly people, with the incidence increasing linearly with each decade above age 40 yrs. It is known that this disease is characterized by the clonal expansion of CD5+ B cells.

MicroRNAs, representing between 1% and 3% of all eukaryotic genes, are a class of endogenous noncoding RNAs, 19-25 nt in size, which regulate gene expression at the transcriptional or translational level. Approximately half of human microRNAs are located at fragile sites and genomic regions involved in alterations in cancers, and alteration of microRNA expression profiles occurs in most cancers, suggesting that individual microRNAs could function as tumor suppressors or oncogenes.

The 13q14 deletion is the most common CLL aberration and is detected by cytogenetic analysis in approximately half of the cases. Analysis of a deletion at 13q14.3 led to the discovery of two physically linked microRNAs, miR-15a and miR-16-1, as targets of these deletions. Consequently, miR-15a and miR-16-1 expression is reduced in the majority of CLL cases, and further studies indicated that miR-15a/miR-16-1 negatively regulate Bc12 expression. These findings indicated that micro-RNAs play important roles in CLL and that down-regulation of miR-15/16 and subsequent Bc12 up-regulation contribute to CLL pathogenesis. Because miR-15/16 was identified as a tumor suppressor in indolent CLL, the microRNA expression profile in CLL has been studied extensively, and a signature profile was reported describing 13 microRNAs that differentiate aggressive and indolent CLL.

miRNA-29 expression is downregulated in aggressive CLL as compared with indolent CLL, and it is believed that miR-29 might function as a tumor suppressor by targeting several oncogenes, including TCL1, MCL1, and CDK6. On the other hand, one report showed that miR-29 expression is up-regulated in metastatic breast cancer, and a very recent study reported that miR-29 overexpression can cause acute myeloid leukemia (AML) in mice.

To clarify the role of miR-29 in B-cell leukemias, we generated transgenic mice overexpressing miR-29 in B cells and now report the phenotype of this mouse model

It would be useful to have effective model to be able to clarify the role of miR-29 in B-cell leukemias.

### SUMMARY

In one aspect, there is provided herein a transgenic animal whose genome comprises: a nucleic acid construct comprising at least one transcriptional regulatory sequence capable of directing expression to B cells operably linked to a nucleic acid sequence encoding miR-29.

In another aspect, there is provided herein a method of producing animals having a lymphoproliferative disorder.

In another aspect, there is provided herein a method of determining the ability of a therapeutic modality to affect a lymphoproliferative disorder.

In another aspect, there is provided herein a transgenic mouse whose genome comprises a nucleic acid sequence encoding a human B-CLL, wherein the sequence is operably linked to a V_{H} promoter and to a IgH-Eₗa enhancer, wherein the transgenic mouse develops an expanded population of CD5⁺ B cells compared to a control mouse.

In another aspect, there is provided herein a transgenic mouse whose genome comprises a nucleic acid sequence encoding a human mi-R29, wherein the sequence is operably linked to a V_{H} promoter and to a IgH-Eμ enhancer, and wherein the transgenic mouse develops a lymphocytic leukemia that exhibits characteristics of human B-CLL.

In another aspect, there is provided herein a transgenic mouse overexpressing miR-29 in B cells and use of such mouse.

In another aspect, there is provided herein a transgenic mice wherein expression of mouse miR-29a/b cluster is controlled by a VH promoter-IgH-4 enhancer, along with humanized renilla green fluorescent protein (hrGFP), and simian virus 40 (SV40) poly(A) site.

In another aspect, there is provided herein a method for evaluating the efficacy of a therapeutic agent used in the treatment of chronic lymphocytic leukemia, comprising determining whether miR-29a is up-regulated, wherein up-regulation of miR-29 is indicative of indolent human B-CLL as compared with aggressive B-CLL and normal CD19+ B cells.

In another aspect, there is provided herein a transgenic mouse whose genome comprises a nucleic acid construct comprising at least one transcriptional regulatory sequence capable of directing expression in B cells of the mouse, wherein the transcriptional regulatory sequence is operably linked to a nucleic acid encoding a miR-29 gene product comprising a nucleotide sequence having at least 90% sequence identity to miR-29, wherein the mouse exhibits a B cell malignancy.

In another aspect, there is provided herein a method of determining whether an agent affects a B cell malignancy.

In another aspect, there is provided herein a method of testing the therapeutic efficacy of an agent in treating a B cell malignancy.

Other systems, methods, features, and advantages of the present invention will be or will become apparent to one with skill in the art upon examination of the following drawings and detailed description. It is intended that all such additional systems, methods, features, and advantages be included within this description, be within the scope of the present invention, and be protected by the accompanying claims.

### BRIEF DESCRIPTION OF THE DRAWINGS

The patent or application file may contain one or more drawings executed in color and/or one or more photographs. Copies of this patent or patent application publication with color drawing(s) and/or photograph(s) will be provided by the Patent Office upon request and payment of the necessary fee.

FIGS. 1A-1F: MiR-29 expression in CLL and production of Ep-miR-29 transgenic founder mice.

FIG. 1A-MiR-29a and FIG. 1B-miR-29b expression in aggressive and indolent CLL.

FIG. 1C: Ep-miR-29 construct.

FIG. 1D-FIG. 1E: Expression of (FIG. 1D) miR 29a and (FIG. 1E) miR-29b in splenic lymphocytes of Ep-miR-29 founders.

FIG. 1F: Expression of GFP in splenic lymphocytes of Ep-miR-29 founders.

FIGS. 2A-2H: Eμ-miR-29 mice develop CLL.

FIGS. 2A-2C: Flow cytometric analysis of miR 29transgenic (Tg) and control lymphocytes isolated from (FIG. 2A) spleen, (FIG. 2B) peripheral blood, and (FIG. 2C) bone marrow.

FIGS. 2D-2F: Analysis of CD5+ B-cell populations in miR-29 transgenic mice and WT controls.

FIG. 2G: Gross pathology of a representative Eμ-miR-29 transgenic mouse showing advanced CLL and a WT control of the same age.

FIG. 2H: Analysis of IgH gene configuration by Southern blot: spleen lymphocyte DNA isolated from five representative cases showing at least 50% CD5+CD19′ B cells. Clonal rearrangements are indicated by asterisks

FIGS. 3A-3L: Histopathological analysis of Eμ-miR-29 mice. Smudge cells indicated by arrowheads. Atypical lymphoid cells are indicated by black arrows. A normal lymphoid follicle is indicated by a green arrow.

FIGS. 4A-4L: Cell-cycle analysis of leukemic cells from Eμ-miR-29 transgenic mice.

FIGS. 4A-4D: BrdU incorporation into DNA of WT B220++ B cells.

FIGS. 4E-4J: BrdU incorporation into transgenic B220+CD5+ and B220+CD5− B-cell DNA.

FIG. 4K: Ig levels in serum of WT and transgenic animals.

FIG. 4L: Levels of anti-SRBC-specific antibodies in serum of WT and transgenic animals 7 d after SRBC injection.

FIGS. 5A-5C: Mir-29 transgene expression accelerates CLL in Eμ-TCL1 mice.

FIG. 5A: Flow cytometric analysis of Eμ-TCL1/4-miR-29 and Eμ-TCL1 transgenic lymphocytes from spleen.

FIG. 5B: Percentage of CD5′ B cells in Eμ-TCL1/4-miR-29 and Eμ-TCL1 transgenic spleen lymphocytes.

FIG. 5C: Spleen weight from Eμ-TCL1/ Eμ-miR-29 and Eμ-TCL1 transgenic mice.

FIGS. 6A-6F: Analysis of miR-29 targets in Eμ-miR-29 transgenic mice.

FIG. 6A: Western blot analysis of Cdk6, DNMT3A, PTEN, and Mc11 expression in CD19+ B cells of miR-29 transgenic and WT mice.

FIG. 6B: Microarray expression data for PXDN, BCL7A, and ITIH5 in CD19+ B cells of miR-29 transgenic and WT mice.

FIG. 6C: Sequence alignments of miR-29a [SEQ ID No: 1] and 3′ UTRs of PXDN [SEQ ID No: 2], BCL7A [SEQ ID No: 3], and ITIH5 [SEQ ID No: 4].

Fig. D: miR-29 targets PXDN but not BCL7A and ITIH5 expression in luciferase assays.

FIG. 6E: Effect of miR-29 on Pxdn protein expression.

FIG. 6F: PDXN expression in CLL.

FIGS. 7A-7L: Histopathological analysis of chronic lymphocytic leukemia (CLL) invasion in liver and kidney of Eμ-miR-29 mice.

### DETAILED DESCRIPTION

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

The present invention is based, at least in part, on the inventors' discovery that clarifies the role of miR-29 in B-cell leukemias.

In a first aspect, there is provided herein a transgenic mice overexpressing miR-29 in B cells; and now reported herein is the phenotype of this mouse model. miR-29a is up-regulated in indolent human B-CLL as compared with aggressive B-CLL and normal CD19+ B cells.

To study the role of miR-29 in B-CLL, the inventors herein generated 4-miR-29 transgenic mice overexpressing miR-29 in mouse B cells. Flow cytometric analysis revealed a markedly expanded CD5+ population in the spleen of these mice starting at 2 mo of age, with 85% (34/40) of miR-29 transgenic mice exhibiting expanded CD5+ B-cell populations, a characteristic of B-CLL. On average, 50% of B cells in these transgenic mice were CD5 positive.

At 2 y of age the mice showed significantly enlarged spleens and an increase in the CD5+ B-cell population to -100%. Of 20 4-miR-29 transgenic mice followed to 24-26 mo of age, 4 (20%) developed frank leukemia and died of the disease. These results show dysregulation of miR-29 can contribute to the pathogenesis of indolent B-CLL.

### EXAMPLES

The present invention is further defined in the following Examples, in which all parts and percentages are by weight and degrees are Celsius, unless otherwise stated. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. All publications, including patents and non-patent literature, referred to in this specification are expressly incorporated by reference. The following examples are intended to illustrate certain preferred embodiments of the invention and should not be interpreted to limit the scope of the invention as defined in the claims, unless so specified.

The value of the present invention can thus be seen by reference to the Examples herein.

Materials and Methods

Eμ-miR-29 Transgenic Mice and Human CLL Samples. A 1.0-kb fragment containing mouse miR-29ab cluster was cloned into the B amHI and SalI sites of the plasmid containing a mouse VH promoter (V186.2) and the IgH-Eμ enhancer along with the hrGFP and the SV40 poly(A) site. The miR-29a/b cluster sequence was inserted within the intron of this construct. Transgenic mice were produced in Ohio State University transgenic mouse facility. Genotyping was performed on tail DNAs by PCR using the primers: miR-29d: get gac gtt gga gcc aca ggt aag [SEQ ID No: 5]; miR-29r: aca aat tcc aaa aat gac ttc cag [SEQ ID No: 6].

Human CLL samples were obtained from the Chronic Lymphocytic Leukemia Research Consortium after informed consent was obtained from patients diagnosed with CLL. Research was performed with the approval of the Institutional Review Board of The Ohio State University. RNA extraction was carried. Real-time PCR experiments were carried out using miR-29a, miR-29b, and PXDN assays for real-time PCR (Applied Biosystems) according to the manufacturer's protocol. Control human cord blood CD19+ B cells were purchased from Allcells and Lonza.

Characterization of miR-29 Transgenic Lymphocytes.

Lymphocytes from spleens and bone marrow were isolated. Flow cytometry measurements of SRBC immune response, Ig levels, and proliferation of B-cell populations were carried out. To analyze IgH gene rearrangements, Southern blot analysis of spleen lymphocyte DNA was carried out using EcoRI digestions and mouse JH4 probe.

For histology and immunohistochemistry, mice were necropsied, and spleens, livers, and kidneys were fixed in 10% buffered formalin, included in paraffin, and then cut in 4-μm sections. Sections were stained with H&E according to standard protocols.

Analysis of miR-29 Targets.

B cells were isolated using a B-cell isolation kit (Miltenyi Biotec) according to the manufacturer's instructions. Proteins from spleens were extracted. Western blot analysis was carried out using Cdk6 (H-96; Santa Cruz Biotechnology), DNMT3A (2160; Cell Signaling Technology), Pten (mmac 1; Lab Vision), Mc11 (S-19; Santa Cruz Biotechnology), Pdxn (Novus), and GAPDH (2118; Cell Signaling Technology) antibodies. For luciferase assays, fragments of PXDN, BCL7A, and ITIH5 cDNA, including regions complimentary to miR-29, were inserted into a pGL3 vector using the XbaI site immediately downstream from the stop codon of luciferase. MiR-29a, miR-29b, and scrambled control RNA duplexes were purchased from Ambion. The expression construct containing full-length human PXDN was purchased from OriGene. Transfections were carried.

Results

MiR-29 Expression in CLL and Production of the Eu-miR-29 Transgenic Mouse Model.

To determine expression levels of miR-29 in CLL and normal CD19+ B cells, the inventors herein studied the expression of miR-29a and miR-29b in 29 aggressive CLL samples, 33 indolent CLL samples, and two normal CD19+ B-cell controls.

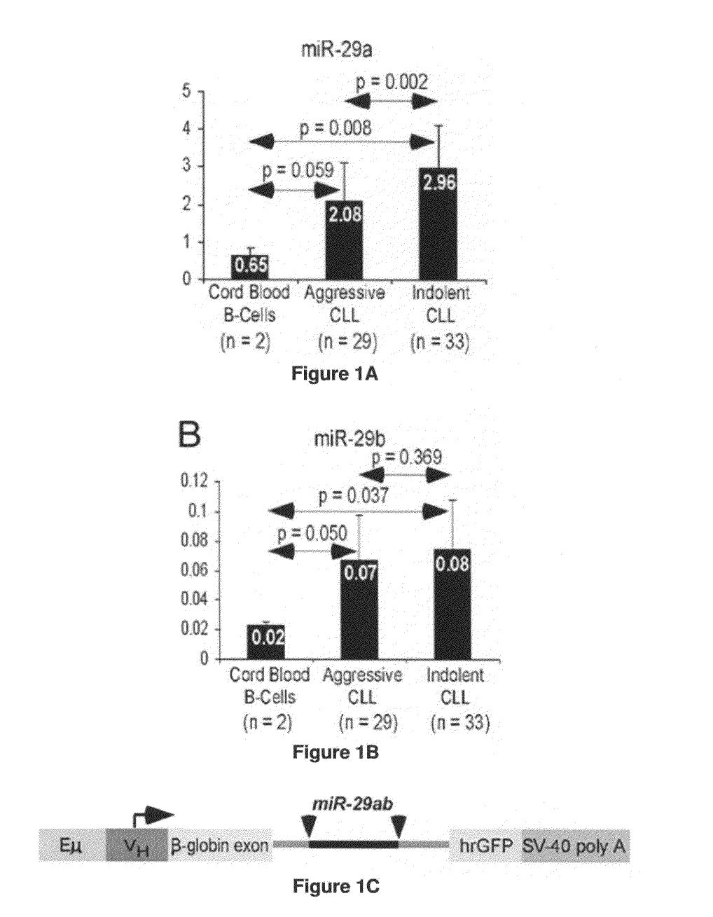

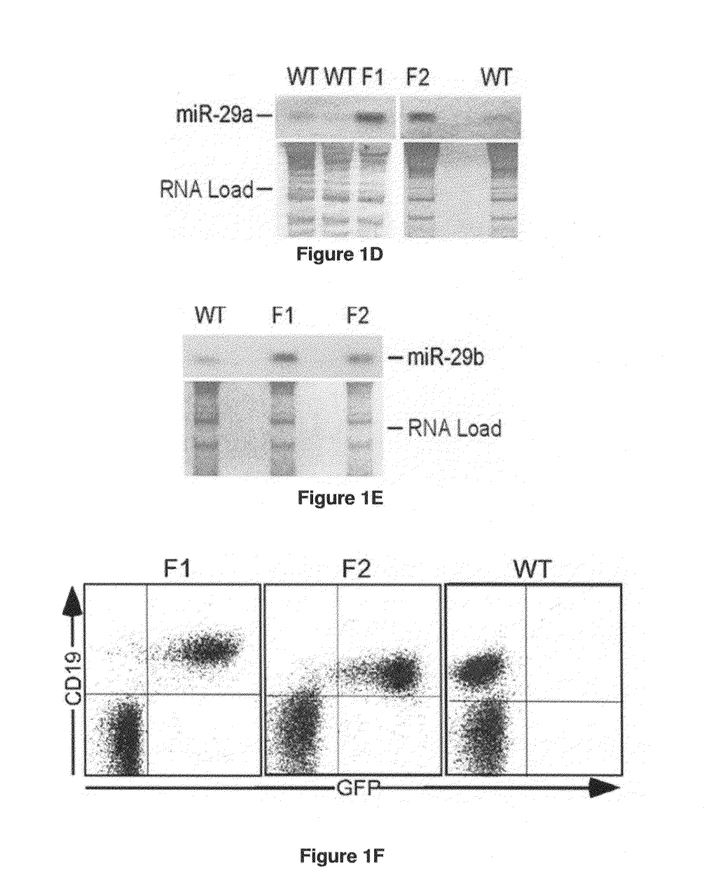

FIG. 1A and FIG. 1B show real-time RT-PCR results in these samples. miR-29a expression was 4.5-fold higher in indolent CLL than in normal CD19+ B cells, whereas aggressive CLL samples showed a 3.2-fold increase. Similarly, miR-29b expression was increased 4-fold in indolent CLL and 3.5-fold in aggressive CLL compared with normal CD19+ B cells. Both miR-29a and miR-29b were down-regulated in aggressive versus indolent CLL, although in the case of miR-29b this difference was not statistically significant (FIG. 1B).

Interestingly, in all samples miR-29a expression level was more than 20-fold higher than that of miR-29b (FIG. 1A and FIG. 1B).

Because expression levels of miR-29a and miR-29b were significantly higher in indolent CLL than in normal CD19+ B cells, the inventors herein now believe that miR-29 may contribute to the pathogenesis of CLL.

To investigate, the inventors herein developed transgenic mice in which expression of the mouse miR-29a/b cluster was controlled by a VH promoter-IgH-4 enhancer, along with humanized renilla green fluorescent protein (hrGFP), and the simian virus 40 (SV40) poly(A) site.

This promoter/enhancer combination drives expression of miR-29a/b in immature and mature B cells (FIG. 1C). The miR-29a/b cluster sequence was inserted within the intron of this construct (FIG. 1C). Two founders on FVB/N background, designated “F1” and “F2,” were generated and bred to establish the transgenic lines. Expression of miR-29a and miR-29b was examined by Northern blot analysis, using RNAs isolated from spleens of transgenic animals.

FIG. 1D and FIG. 1E show overexpression of miR-29a and miR-29b in both transgenic lines (F1 and F2) compared with nontransgenic (WT) siblings. To confirm that the transgene is expressed in B cells, the inventors performed flow cytometry using CD19 as a B-cell marker.

FIG. 1F shows that all CD19+ cells in both transgenic lines also express GFP (F1 and F2), whereas no GFP expression was detected in WT littermates.

Ett-miR-29 Transgenic Mice Show CLL Phenotype.

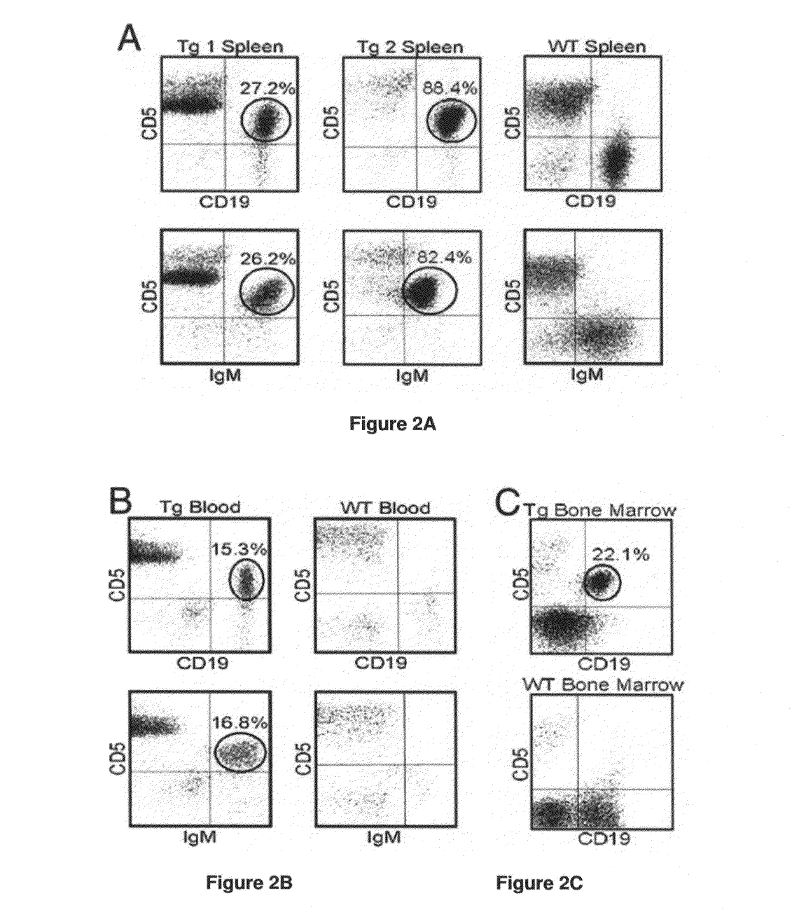

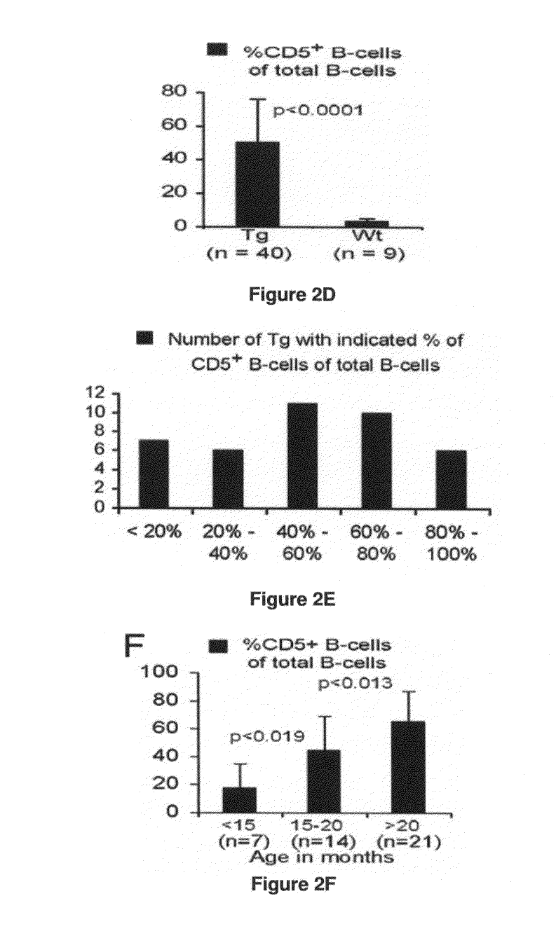

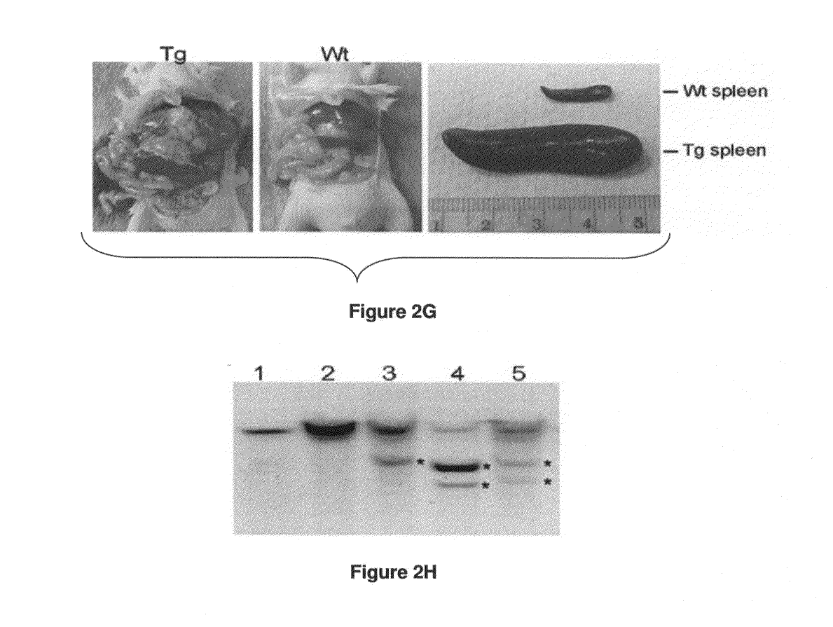

Flow cytometry was used to determine the immunophenotypic profile of spleen lymphocytes from miR-29 transgenic mice. At the age of 12-24 mo, flow cytometric analysis revealed a markedly expanded CD5+ B-cell population (a characteristic of CLL) in the spleen of 34 of 40 (85%) miR-29 transgenic mice; −50% of B cells in these transgenic mice were CD5+. FIG. 2A (Left) shows a representative example. Although almost all spleen B cells from this animal were CD5+CD19+IgM+, these cells represented only 25-30% of all spleen lymphocytes. A more advanced CLL case is shown in FIG. 2A (Center). Almost all normal lymphocytes in the spleen of this animal were replaced by malignant CD5+CD19+IgM+ B cells. Almost no CD5+CD19+IgM+ B cells were detected in spleens of WT littermates (FIG. 2A, Right).

The expanded population of CD5+CD19+ B cells also was detected in peripheral blood and bone marrow from miR-29 transgenic mice, but not from WT littermates (FIG. 2B and FIG. 2C).

FIGS. 2D-2F show the number of animals with increased CD5+CD19+IgM+ populations in spleen. Although only 7 of 40 (17%) miR-29 transgenic mice showed 0-20% CD5+ B cells, 16 of 40 (40%) showed 60% or more CD5+CD19+IgM+ cells. In addition, miR-29 transgenic mice showed significant increases in the percentage of CD5+ splenic B cells with age (FIG. 2F). In animals younger than 15 mo, CD5+ B cells represented only −20% of total B cells; by 15-20 mo of age, that percentage increased to −40% (FIG. 2F).

At the age of 20-26 mo, on average, >65% of all B cells were CD5+ (FIG. 2F). These data show gradual progression of indolent CLL in miR-29 transgenic mice. Twenty Eμ-miR-29 mice were followed to the age of 24-26 mo. Almost all these mice showed significantly enlarged spleens, and 4 of 20 (20%) developed frank leukemia and died of disease. FIG. 2G shows a representative case of frank leukemia presenting with an enlarged spleen and liver and advanced lymphadenopathy.

Clonal IgH gene rearrangements are typical in human CLL cases. These rearrangements also were observed in the Tcll-driven mouse model of CLL. To determine if CD5+ B cells from Eμ-miR-29 transgenic mice show clonality, Southern blot hybridization were carried using spleen lymphocyte DNA isolated from cases showing at least 50% CD5+CD19+IgM+ B cells. FIG. 2H shows clonal rearrangements of the IgH gene in three of five cases analyzed. These results further indicate that the expansion of CD5+ B cells in Ep-miR-29 mice resembles human CLL.

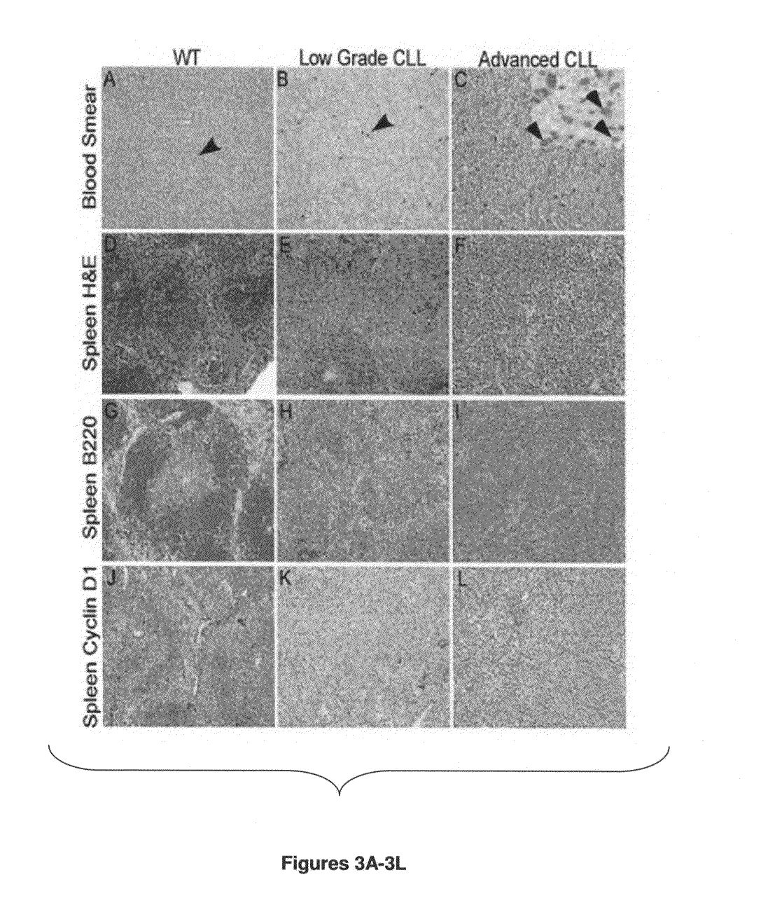

To confirm further that Ep-miR-29 mice develop CLL-like disease, histological and immunohistological analysis were carried out. FIGS. 3 A-3C shows representative smears from blood of Ep-miR-29 transgenic mice and a WT control. The smear from a WT mouse showed rare lympho-monocytes with a normal appearance (FIG. 3A). In contrast, the smear from a Eμ-miR-29 mouse with low-grade CLL exhibited an increased number of atypical lymphoid cells (FIG. 3B, black arrows), and the smear from a miR-29 transgenic mouse with advanced CLL presented numerous malignant lymphoid cells (FIG. 3C), including smudge cells, typical of CLL (FIG. 3C, Inset; smudge cells are indicated by arrowheads).

FIGS. 3D-3L show representative histological images of Ep-miR-29 transgenic mice and a WT control. The spleen of the WT mouse shows preserved architecture and several normal-looking lymphoid follicles (FIG. 3D, green arrow). In contrast, the spleen of a diseased miR-29 transgenic mouse with CLL exhibits distorted architecture (FIG. 3E), and the spleen of a miR-29 mouse with advanced CLL shows total obliteration of the normal architecture by malignant lymphoid proliferation (FIG. 3F).

B220 staining of the same sections shows a lymphoid follicle of a WT mouse presenting a normal B-cell disposition (FIG. 3G). In contrast, transgenic spleens show lymphoid follicles in disarray because of the low-grade malignant lymphoid proliferation (FIG. 3H) or CLL with diffuse distribution of a B-cell malignant population (FIG. 31).

FIGS. 3J-3L shows low expression of cyclin D1 in a WT spleen (FIG. 3J) and moderate to high cyclin D1 expression in low-grade CLL (FIG. 3K) and advanced CLL (FIG. 3L). Thus, the histological and immunohistological examination confirmed that Ep-miR-29 mice develop CLL-like disease.

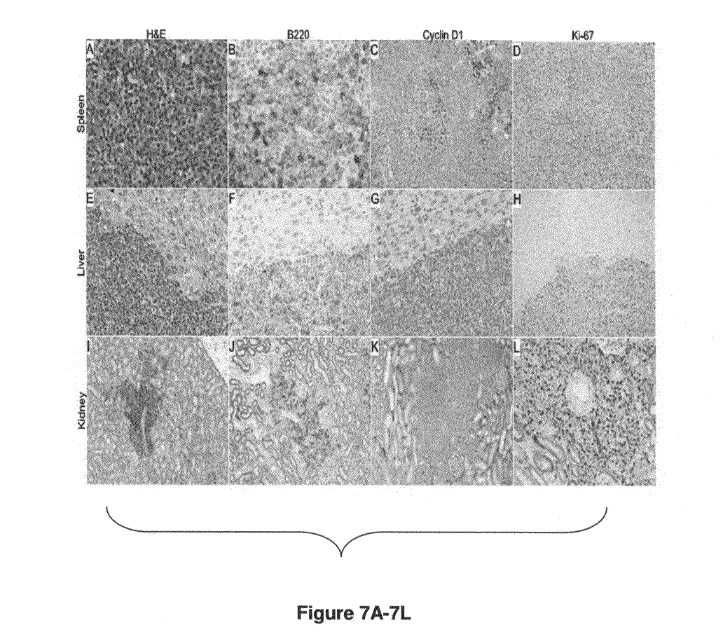

As noted above, only 20% of Ep-miR-29 transgenic mice developed advanced leukemia and died from the disease. FIGS. 7A-7L show a representative advanced case of CLL that invaded liver and kidney. Histological examination showed total obliteration of the normal spleen architecture with high expression of B220, cyclin D1, and Ki67 (FIGS. 7A-7D).

These B220+ malignant B cells invaded liver (FIGS. 7E-7H) and kidney (FIGS. 71-7L).

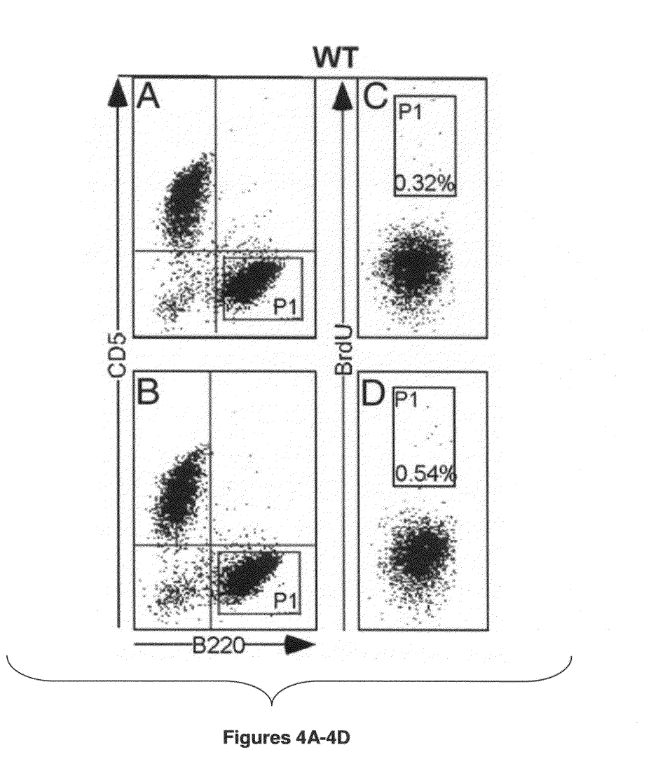

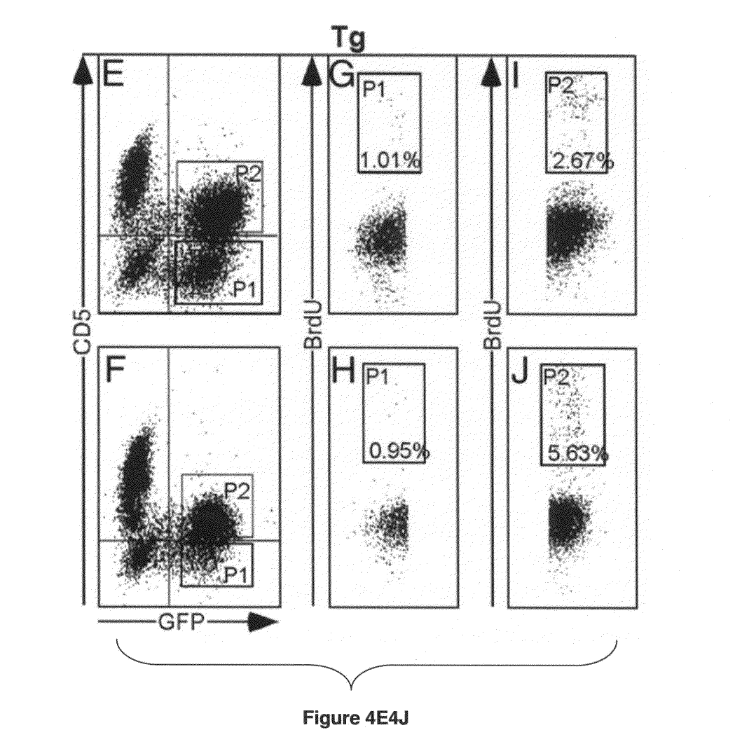

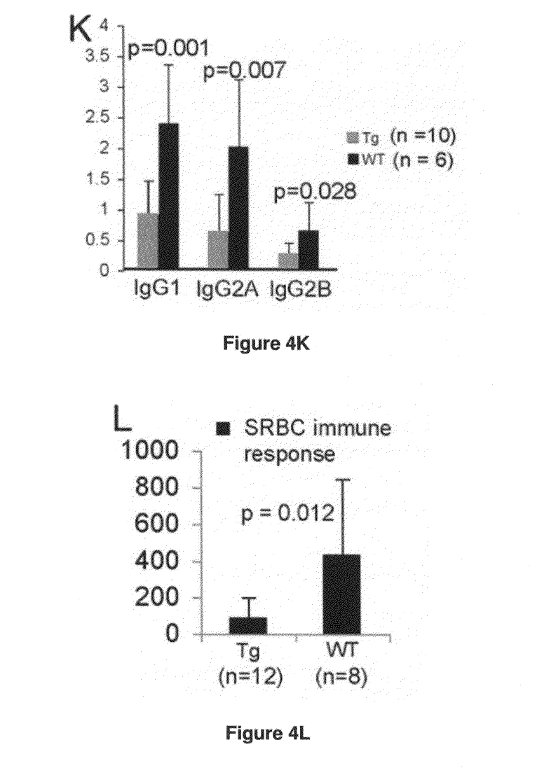

Accumulation of CLL lymphocytes can result not only from prolonged survival, but also from proliferating CD5+B220+ cells originating in the bone marrow, lymph nodes, or spleen. Therefore, to determine whether CLL cells from Ep-miR-29 mice proliferate, the inventors herein used cell cycle analyses based on BrdU incorporation. The inventors assessed the proliferative capacity of B220+CD5+, as well as B220+CD5− transgenic splenic lymphocytes in comparison with WT B220+ splenic lymphocytes. FIGS. 4A-4J shows that B220+CD5+ B cells from Eμ-miR-29 mice proliferate, whereas no proliferation was detected for B220+ WT lymphocytes (2.7% and 5.6% cells in S-phase for transgenic B cells versus 0.3% and 0.5% for WT B cells (FIGS. 41-4J versus FIGS. 4C-4D). Interestingly, even B220+CD5− transgenic lymphocytes showed increased proliferation compared with B220+ WT B cells, with 1.0% and 0.95% cells in S-phase versus 0.3% and 0.5% for WT B cells (FIGS. 4G-4H versus FIGS. 4C-4D).

These data show that miR-29 overexpression promotes B-cell proliferation, even in CD5⁻ cells. Human CLL is characterized by immune incompetence and progressive severe hypogammaglobulinemia that eventually develops in almost all patients. Therefore, to determine if Eμ-miR-29 mice develop hypogammaglobulinemia, the inventors herein compared levels of serum Ig in transgenic mice and in WT littermates at age −18 mo.

FIG. 4K shows that the levels of IgG1, IgG2a, and IgG2b were decreased 2- to 4-fold in Eμ miR 29 transgenic mice as compared with WT controls. To determine if Eμ-miR-29 mice show impaired immune response, the inventors compared levels of anti-sheep RBC (SRBC) antibodies after injection of SRBC in miR-29 transgenic mice and WT siblings. FIG. 4L shows that serum levels of anti-SRBC antibodies were decreased -4-fold in serum of miR-29 transgenic mice compared with age-matched WT mice. These data clearly indicate that, as in human CLL, the CLL-like disease in Eμ-miR-29 mice is characterized by hypogammaglobulinemia and immune incompetence.

In the instant mouse model described herein, the TCL1 ORF (lacking 3′ UTR) was under the control of a VH promoter-IgH-Eμ enhancer. Because of the absence of the 3′ UTR in the transgenic construct, miR-29 could not inhibit TCL1 expression in these mice. Eμ-TCL1 transgenic mice develop aggressive CLL, and all mice die of the disease at 12-15 mo of age. To determine if transgenic miR-29 expression can accelerate CLL in ERTCL1 transgenic mice, the inventors herein crossed Eμ-miR-29 and Eμ-TCL1 transgenic mice. Eμ-miR-29/4t-TCL1 mice and their Eμ-TCL1 littermates were killed at −8 mo of age and analyzed.

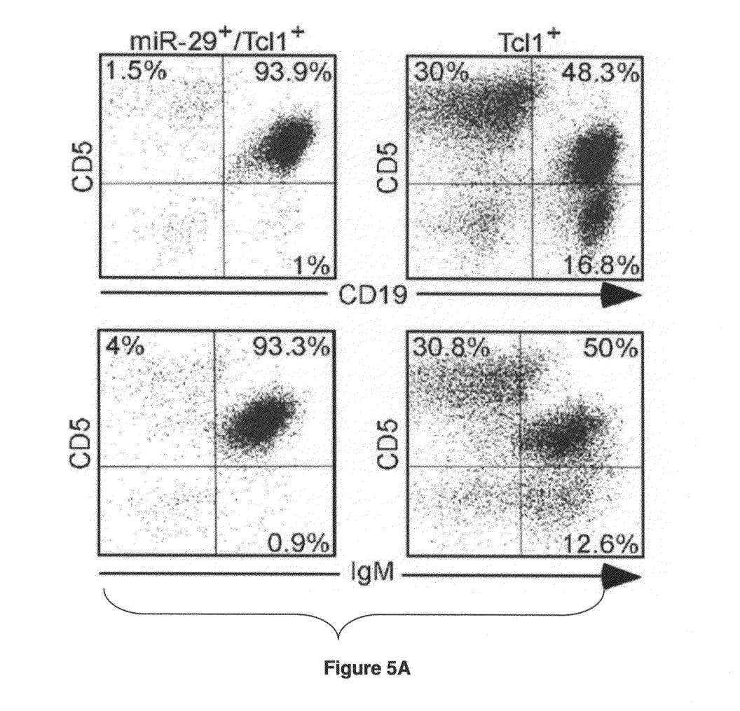

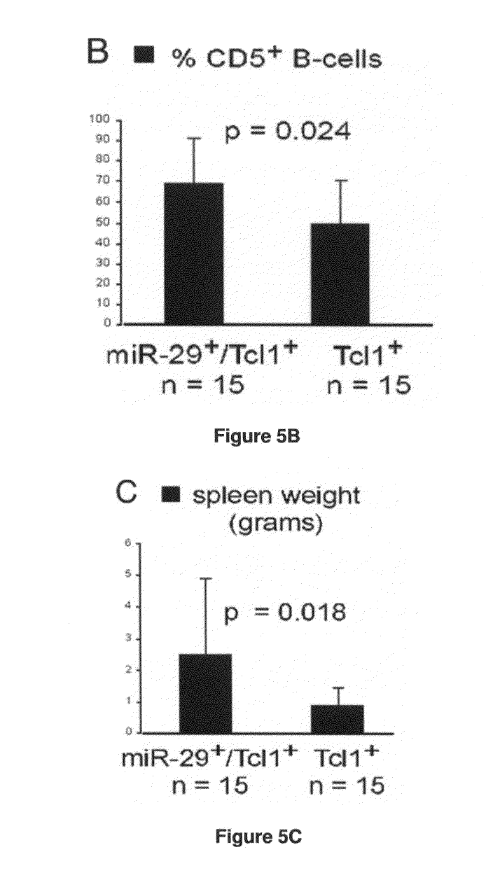

FIG. 5A shows representative FACS analysis of spleen lymphocytes of these genotypes. TCL1/miR-29 double transgenic mice showed significantly increased CD5+CD19+ and CD5+IgM+ B-cell populations compared with Eμ-TCL1 mice (93.9% and 93.3% versus 48.3% and 50%). On average, Eμ-miR-29/4t-TCL1 mice had 40% more CD5+CD19++ splenic B cells and 3-fold increases in spleen weight compared with Eₗl-TCL1 mice (FIGS. 5B-5C). These data show that miR-29 can contribute to the pathogenesis of CLL independently of Tell.

Analysis of miR-29 Targets.

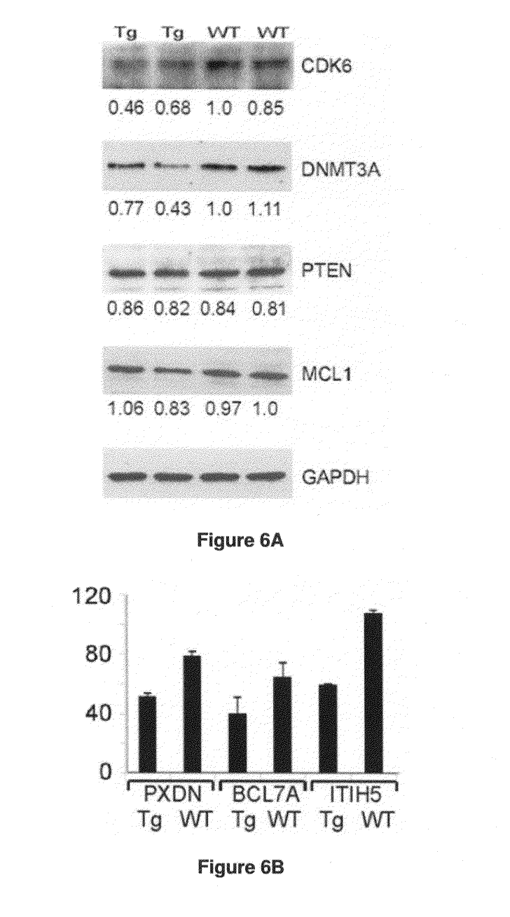

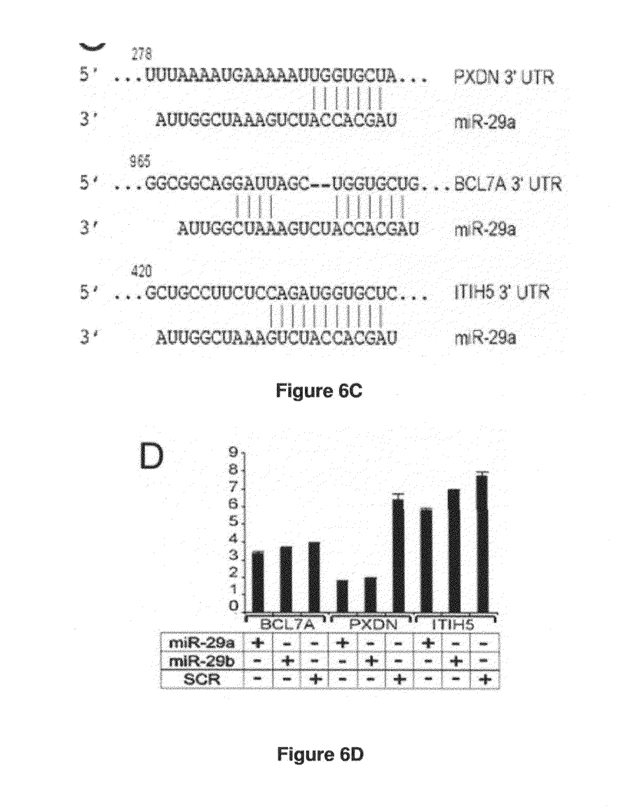

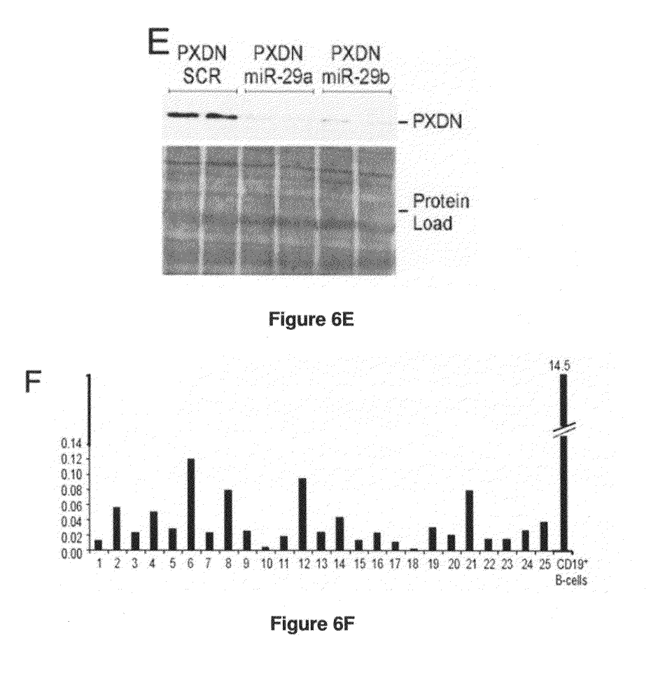

To determine whether miR-29 over-expression in mouse B cells affects expression of its targets, the expression levels of several previously reported miR-29 targets, Cdk6, Men, and DNMT3A were analyzed, in sorted B220+ B cells from miR-29 transgenic mice and WT controls. It was then found that two targets, Cdk6 and DNMT3A, are down-regulated in miR-29 transgenic mice, whereas no differences in Mc11 and Pten were detected (FIG. 6A) [although Pten is not a proven miR-29 target, it previously have been predicted to be a potential target].

Because Cdk6 and DNMT3 are not known to be tumor suppressors, Affymetrix gene expression arrays were used to determine potential miR-29targets contributing to its oncogenic activity. Using microarray analysis, the gene expression was compared in sorted B220+ B cells from miR-29 transgenic mice and WT controls. The inventors then cross-referenced genes down-regulated in miR-29 transgenic B cells that had known or potential tumor suppressor function with the list of potential miR-29 targets obtained from Targetscan software. Three potential targets were identified: peroxidasin (PXDN), a p53-responsive gene down-regulated in AML; Bc17A, a proapoptoticgenedown-regulatedin T-celllymphomas; and ITIH5, a member of the inter-a-trypsin inhibitor family down-regulated in breast cancer.

FIGS. 6B-6C show the down-regulation of expression of these three genes in CD19+ B cells of miR-29 transgenic mice versus WT littermates and the alignment of miR-29a and corresponding 3′ UTRs. To determine if miR-29 indeed targets expression of PXDN, Bc17A, and ITIH5, the 3T UTR fragments (including miR-29 homology regions) of these cDNAs were inserted downstream of the luciferase ORF into pGL3 vector. HEK293 cells were cotransfected with miR-29a, miR-29b, or scrambled negative control and a pGL3 construct containing fragments of PXDN, Bc17A, and ITIH5 cDNAs, including a region homologous to miR-29, as indicated (FIG. 6D).

Expression of miR-29a or miR-29b significantly (-3-fold) decreased luciferase expression of the construct containing the 3′ UTR of PXDN, whereas no significant effect was observed for Bc17A and ITIH5 (FIG. 6D). Thus, while not wishing to be bound by theory, the inventors herein now believe that PXDN expression may be targeted by miR-29. To confirm, full-length PXDN cDNA including 5′ and 3′ UTRs were used in a cytomegalovirus mammalian expression vector and investigated whether miR-29 expression affects Pdxn protein expression levels.

This construct was cotransfected with miR-29a, miR-29b, or PremiR negative control (scrambled) into HEK293 cells, as indicated in FIG. 6E. These experiments revealed that coexpression of PXDN with miR-29a or miR-29b almost completely inhibited Pxdn expression (FIG. 6E). The inventors herein now believe that miR-29a and miR-29b target Pxdn expression at mRNA and protein levels. To determine if Pdxn plays a role in the pathogenesis of human CLL, the expression of PXDN in 25 human CLL samples and normal CD19+ B-cell controls was studied.

FIG. 6F shows real-time RT-PCR results in these samples. PXDN expression was drastically down-regulated *50-fold or more) in CLL samples compared with normalCD19B cells. These results show that the oncogenic role of miR-29 in B cells might be, at least in part, dependent on targeting peroxidasin.

Discussion

The present invention shows that miR-29 over-expression in B cells results in CLL and that miR-29 is overexpressed in indolent CLL compared with normal B cells.

Because only 20% of Eμ-miR-29 transgenic mice died of leukemia in old age, but almost all mice showed expanded CD5+CD19+ B-cell populations, the phenotype of Eμ-miR-29 is similar to that of indolent CLL. Therefore up-regulation of miR-29 initiates or at least significantly contributes to the pathogenesis of indolent CLL. On the other hand, TCL1 is mostly not expressed in indolent CLL and probably does not play an important role in indolent CLL.

While not wishing to be bound by theory, the inventors herein now believe is that miR-29 overexpression is not sufficient to initiate aggressive CLL. In contrast, up-regulation of Tc11 is a critical event in the pathogenesis of the aggressive form of CLL. Because miR-29 targets TCL1, its down-regulation in aggressive CLL (compared with the indolent form) contributes to up-regulation of Tc11 and the development of an aggressive phenotype.

While the invention has been described with reference to various and preferred embodiments, it should be understood by those skilled in the art that various changes may be made and equivalents may be substituted for elements thereof without departing from the essential scope of the invention. In addition, many modifications may be made to adapt a particular situation or material to the teachings of the invention without departing from the essential scope thereof.

Therefore, it is intended that the invention not be limited to the particular embodiment disclosed herein contemplated for carrying out this invention, but that the invention will include all embodiments falling within the scope of the claims.

1. A transgenic animal whose genome comprises: a nucleic acid construct comprising at least one transcriptional regulatory sequence capable of directing expression to B cells operably linked to a nucleic acid sequence encoding miR-29.

2. The transgenic animal of claim 1 wherein the at least one transcriptional regulatory sequence comprises a V_{H} promoter.

3. The transgenic animal of claim 2 wherein the at least one transcriptional regulatory sequence further comprises a IgH-Eμ enhancer.

4. The transgenic animal of claim 1 wherein the nucleic acid sequence encoding miR-29 comprises a DNA sequence encoding human miR-29.

5. The transgenic animal of claim 2 wherein the V_{H} promoter is derived from mouse.

6. The transgenic animal of claim 3 wherein the IgH-Eμ enhancer is derived from mouse.

7. The transgenic animal of claim 1 wherein the animal is a mouse.

8. The transgenic animal of claim 1 wherein the animal exhibits an expanded population of CDS⁺ B cells.

9. The transgenic animal of claim 1 wherein the animal exhibits a lymphoproliferative condition.

10. The transgenic animal of claim 9 wherein the lymphoproliferative condition comprises a preleukemic state.

11. The transgenic animal of claim 9 wherein the lymphoproliferative condition comprises leukemia.

12. The transgenic animal of claim 11 wherein the leukemia exhibits characteristics of human B-CLL.

13. A transgenic animal whose genome comprises a nucleic acid construct comprising a nucleic acid sequence encoding miR-29, wherein the sequence is operably linked to a V_{H} promoter and to a IgH-En enhancer, wherein miR-29 is expressed in immature and mature B cells of the animal.

14. A method of producing animals having a lymphoproliferative disorder comprising the steps of:
a) obtaining white blood cells from a transgenic animal whose genome comprises: a nucleic acid construct comprising at least one transcriptional regulatory sequence capable of directing expression to B cells operably linked to a nucleic acid sequence encoding miR-29;
b) counting the cells; and,
c) injecting a number of the cells into a recipient animal syngeneic with the transgenic animal, wherein the number of the cells so injected is effective to produce a lymphoproliferative disorder in the recipient animal.

15. A method of determining the ability of a therapeutic modality to affect a lymphoproliferative disorder, the method comprising the steps of :
a) providing a first transgenic animal whose genome comprises: a nucleic acid construct comprising at least one transcriptional regulatory sequence capable of directing expression to B cells operably linked to a nucleic acid sequence encoding miR-29;
b) administering the therapeutic modality to the first transgenic animal;
c) performing an analysis of the population of B cells in the transgenic animal;
d) providing a control animal, wherein the control animal is a second transgenic animal whose genome comprises: a nucleic acid construct comprising at least one transcriptional regulatory sequence capable of dⁱrecting expression to B cells operably linked to a nucleic acid sequence encoding miR-29, wherein the control animal does not receive the therapeutic modality;
e) performing an analysis of the population of B cells in the control animal; and, f) comparing the analysis of step c) with the analysis of step e), wherein the ability of the therapeutic modality to affect a lymphoproliferative disorder is evidenced by a difference in the B cell population between the first transgenic animal and the control animal.

16. The method of claim 15 wherein the lymphoproliferative disorder comprises a B cell neoplasia.

17. The method of claim 16 wherein the B cell neoplasia is B-CLL.

18. The method of claim 15 wherein the first transgenic animal and the control animal are mice.

19. The method of claim 18 wherein the analysis comprises a measurement of the number and/or relative proportion of CDS⁺ B cells.

20. A transgenic mouse whose genome comprises a nucleic acid sequence encoding a human B-CLL, wherein the sequence is operably linked to a V_{H} promoter and to a IgH-En enhancer, wherein the transgenic mouse develops an expanded population of CD5+ B cells compared to a control mouse.

21. The transgenic mouse of claim 20, wherein the V_{H} promoter comprises a mouse V_{H} promoter.

22. The transgenic mouse of claim 20, wherein the IgH-En enhancer comprises a mouse IgH-En enhancer.

23. The transgenic mouse of claim 20, wherein the mouse develops a lymphocytic leukemia which exhibits characteristics of human B-CLL.

24. A transgenic mouse whose genome comprises a nucleic acid sequence encoding a human mi-R29, wherein the sequence is operably linked to a V_{H} promoter and to a IgH-En enhancer, and wherein the transgenic mouse develops a lymphocytic leukemia that exhibits characteristics of human B-CLL.

25. The transgenic mouse of claim 24, wherein the V_{H} promoter comprises a mouse V_{H} promoter.

26. The transgenic mouse of claim 24, wherein the IgH-En enhancer comprises a mouse IgH-En enhancer.

27. A transgenic mouse overexpressing miR-29 in B cells.

28. (canceled)

29. A transgenic mice wherein expression of mouse miR-29a/b cluster is controlled by a VH promoter-IgH-En enhancer, along with humanized renilla green fluorescent protein (hrGFP), and simian virus 40 (SV40) poly(A) site.

30. A method for evaluating the efficacy of a therapeutic agent used in the treatment of chronic lymphocytic leukemia, comprising determining whether miR-29a is up-regulated, wherein up-regulation of miR-29 is indicative of indolent human B-CLL as compared with aggressive B-CLL and normal CD19+ B cells.

31. A transgenic mouse whose genome comprises a nucleic acid construct comprising at least one transcriptional regulatory sequence capable of directing expression in B cells of the mouse, wherein the transcriptional regulatory sequence is operably linked to a nucleic acid encoding a mi-R29 gene product comprising a nucleotide sequence having at least 90% sequence identity to miR-29, wherein the mouse exhibits a B cell malignancy.

32. The transgenic mouse of claim 31, wherein the at least one transcriptional regulatory sequence comprises a V_{H} promoter.

33. The transgenic mouse of claim 31, wherein the at least one transcriptional regulatory sequence comprises an IgH-Eμ enhancer.

34. The transgenic mouse of claim 31, wherein the nucleic acid encodes a miR-29 gene product comprising [SEQ ID No:1].

35. The transgenic mouse of claim 32, wherein the V_{H} promoter is derived from mouse.

36. The transgenic mouse of claim 33, wherein the IgH-Eμ enhancer is derived from mouse.

37. The transgenic mouse of claim 31, wherein the B cell malignancy is a leukemia, lymphoma or neoplasm.

38. The transgenic mouse of claim 31, wherein the B cell malignancy exhibits characteristics of human acute lymphoblastic leukemia, human lymphoblastic lymphoma or a combination thereof.

39. A method of determining whether an agent affects a B cell malignancy, comprising:
a) administering the agent to a transgenic mouse whose genome comprises a nucleic acid construct comprising at least one transcriptional regulatory sequence capable of directing expression in B cells of the mouse, operably linked to a nucleic acid encoding a miR-29 gene product, wherein the mouse exhibits a B cell malignancy; and
b) after the agent has been administered to the transgenic mouse, comparing one or more symptoms and/or indications of the B cell malignancy in the mouse to those of a control mouse of the same genotype, wherein the control mouse has not been administered the agent, wherein a difference in the detectability and/or rate of appearance of the one or more symptoms and/or indications of the B cell malignancy in the transgenic mouse, relative to the control mouse, is indicative of the agent affecting the B cell malignancy.

40. A method of testing the therapeutic efficacy of an agent in treating a B cell malignancy, comprising:
a) administering the agent to a transgenic mouse whose genome comprises a nucleic acid construct comprising at least one transcriptional regulatory sequence capable of directing expression in B cells of the mouse, operably linked to a nucleic acid encoding a miR-29 gene product, wherein the mouse exhibits a B cell malignancy; and
b) after the agent has been administered to the transgenic mouse, comparing one or more symptoms and/or indications of the B cell malignancy in the mouse to those of a control mouse of the same genotype, wherein the control mouse has not been administered the agent, wherein if the agent inhibits, prevents and/or reduces the one or more symptoms and/or indications of the B cell malignancy in the mouse, relative to the control mouse, then the agent is considered to have therapeutic efficacy in treating or preventing a B cell malignancy.

41. The method of claim 40, wherein the at least one transcriptional regulatory sequence comprises a V_{H} promoter, an IgH-Eμ enhancer or a combination thereof.

42. The method of claim 40, wherein the transcriptional regulatory sequence is derived from mouse.

43. The method of claim 40, wherein the B cell malignancy is selected from the group consisting of acute lymphoblastic leukemia, B cell lymphoma, B cell neoplasm and a combination thereof.

44. The method of claim 41, wherein the B cell malignancy exhibits characteristics of human acute lymphoblastic leukemia, human lymphoblastic lymphoma or a combination thereof.

45. The method of claim 41, wherein the at least one transcriptional regulatory sequence comprises a V_{H} promoter, an IgH-Eμ enhancer or a combination thereof.

46. The method of claim 41, wherein the transcriptional regulatory sequence is derived from mouse.

47. The method of claim 41, wherein the B cell malignancy is selected from the group consisting of acute lymphoblastic leukemia, B cell lymphoma, B cell neoplasm and a combination thereof.

48. The method of claim 42, wherein the B cell malignancy exhibits characteristics of human acute lymphoblastic leukemia, human lymphoblastic lymphoma or a combination thereof.


SEQUENCE LISTING (6 sequences)

SEQ ID NO: 1 - RNA, 22 nt, Artificial Sequence
  Description of Artificial Sequence Synthetic oligonucleotide
uagcaccaucugaaaucgguua

SEQ ID NO: 2 - RNA, 23 nt, Homo sapiens
uuuaaaaugaaaaauuggugcua

SEQ ID NO: 3 - RNA, 23 nt, Homo sapiens
ggcggcaggauuagcuggugcug

SEQ ID NO: 4 - RNA, 23 nt, Homo sapiens
gcugccuucuccagauggugcuc

SEQ ID NO: 5 - DNA, 24 bp, Artificial Sequence
  Description of Artificial Sequence Synthetic primer
gctgacgttggagccacaggtaag

SEQ ID NO: 6 - DNA, 24 bp, Artificial Sequence
  Description of Artificial Sequence Synthetic primer
acaaattccaaaaatgacttccag